(19) United States
(12) Patent Application Publication
(10) Pub. No.: US 2013/0023737 A1
(43) Pub. Date: Jan. 24, 2013

(54) NON-INVASIVE DETECTING APPARATUS AND OPERATING METHOD THEREOF

(71) Applicants: Chung-Cheng Chou, Taoyuan County (TW); William Wang, Taoyuan City (TW); Chung-Ping Chuang, Taoyuan City (TW); Meng-Shin Yen, Taipei City (TW)

(72) Inventors: Chung-Cheng Chou, Taoyuan County (TW); William Wang, Taoyuan City (TW); Chung-Ping Chuang, Taoyuan City (TW); Meng-Shin Yen, Taipei City (TW)

(21) Appl. No.: 13/550,851

(22) Filed: Jul. 17, 2012

(30) Foreign Application Priority Data
Jul. 20, 2011 (TW) 100125575

(51) Int. Cl. A61B 5/00

(52) U.S. Cl. 600/300

(57) ABSTRACT

A non-invasive detecting apparatus and an operating method thereof are disclosed. The non-invasive detecting apparatus includes an elastic base, a detecting module, and a data processing module. The detecting module is disposed on the elastic base. The detecting module includes at least one detecting unit used to detect a tissue under a detected region of a detected object to obtain a detection information. The data processing module analyzes and processes the detection information to generate a detection result.

### BACKGROUND OF THE INVENTION

1. Field of the Invention

The invention relates to medical detection; in particular, to a non-invasive detecting apparatus and an operating method thereof applied in medical wearable non-invasive diagnosis.

2. Description of the Prior Art

With the development of medical technology, various medical detecting and diagnosis apparatuses with different functions are appeared on the market. The conventional invasive medical detecting apparatus will cause pain and discomfort of the tested one, therefore, the conventional invasive medical detecting apparatus has been replaced by a new generation of non-invasive detecting apparatus.

In general, the non-invasive detecting apparatus has advantages of reducing pain and discomfort of the tested one, rapidly obtaining detection result, avoiding pollution; for example, the widely used ultrasound medical detection. Because the current ultrasound medical detection apparatus uses the ultrasound detector of fixed form to detect the tissue under the detected region, when the surface relief of the detected region is too large, the size of the ultrasound detector of the current ultrasound medical detection apparatus or the detection direction of the ultrasound detector should be changed to obtain better detection effect. However, various ultrasound detectors of different sizes should be prepared at the same time, so that it is complicated and cost-consuming, and only a partial region of the ultrasound detector can perform the detection. It is inconvenient for the testing personnel to perform actual detection.

Therefore, the invention provides a non-invasive detecting apparatus and an operating method thereof to solve the above-mentioned problems occurred in the prior arts.

### SUMMARY OF THE INVENTION

An embodiment of the invention is a non-invasive detecting apparatus. In this embodiment, the non-invasive detecting apparatus includes an elastic base, a detecting module, and a data processing module. The detecting module is disposed on the elastic base. The detecting module includes at least one detecting unit used to detect a tissue under a detected region of a detected object to obtain a detection information. The data processing module analyzes and processes the detection information to generate a detection result.

In practical applications, the elastic base can be worn on a hand of a user, and a position of the at least one detecting unit can be changed with the variation of a gesture or the moving of a palm or a finger. The at least one detecting unit can be formed by an emitting unit and a receiving unit, and the emitting unit and the receiving unit are integrated into a transceiver or separated from each other.

In addition, the non-invasive detecting apparatus can further include a positioning module. The positioning module detects a position of the at least one detecting unit, and generates a position compensating information to the data processing module according to a position change of the at least one detecting unit or a relative position change of the at least one detecting unit relative to the elastic base. The data processing module adjusts the detection result according to the position compensating information.

In practical applications, the positioning module and the at least one detecting unit of the detecting module are integrated. The detecting module can use a non-invasive detecting technology to perform the detection. The non-invasive detecting technology can be an ultrasound detecting technology, an optical detecting technology, an electrical detecting technology, or a magnetic detecting technology.

Another embodiment of the invention is a non-invasive detecting apparatus operating method. In this embodiment, the non-invasive detecting apparatus includes an elastic base, a detecting module, and a data processing module. The method includes steps of the at least one detecting unit of the detecting module performing a detection on a tissue under a detected region of a detected object to obtain a detection information; the data processing module analyzing and processing the detection information to generate a detection result.

In practical applications, the method can further include steps of: detecting a position of the at least one detecting unit; generating a position compensating information to the data processing module according to a position change of the at least one detecting unit or a relative position change of the at least one detecting unit relative to the elastic base; adjusting the detection result according to the position compensating information.

Compared to the prior art, the non-invasive detecting apparatus and the operating method thereof disclosed in this invention can avoid the disadvantages of the conventional non-invasive detecting apparatus that various ultrasound detectors of different sizes should be prepared and only a partial region of the ultrasound detector can detect. Even the surface relief of the detected region is too large, since the non-invasive detecting apparatus of the invention can be worn on the hands of the operator, it can be easily operated and smoothly detect without changing detector. It can be also applied in large-area and multi-angles synchronous detection and different non-invasive detecting technologies.

In addition, because the detecting module in the non-invasive detecting apparatus of the invention is integrated with the elastic base, the operator can change the positions of the detecting units (e.g., bending) or the relative positions of the detecting units relative to the elastic base (e.g., shifting) to change the range covered by its detected area. Therefore, compared to the fixed design of detecting units in the detector of the prior art, the detecting module in the non-invasive detecting apparatus of the invention has advantages of high efficiency and high using flexibility.

The advantage and spirit of the invention may be understood by the following detailed descriptions together with the appended drawings.

### BRIEF DESCRIPTION OF THE APPENDED DRAWINGS

FIG. 1 illustrates a function block diagram of the non-invasive detecting apparatus in the first embodiment of the invention.

FIG. 2A illustrates a schematic diagram of the non-invasive detecting apparatus designed in a form of glove.

FIG. 2B, FIG. 2C, and FIG. 2D respectively illustrate detecting motions of bending finger, extending finger, and shifting finger respectively.

FIG. 3A and FIG. 3B illustrate schematic diagrams of the different types of detecting units of the detecting module respectively.

FIG. 4 and FIG. 5 illustrate a function block diagram and the schematic diagram of the non-invasive detecting apparatus in the second embodiment of the invention respectively.

FIG. 6A and FIG. 6B illustrate a side-view and a bottom-view of the positioning module and the detecting unit of the detecting module integrated in the transceiver interface unit.

FIG. 7 illustrates the flowchart of the non-invasive detecting apparatus operating method in the third embodiment of the invention.

FIG. 8 illustrates the flowchart of the non-invasive detecting apparatus operating method in the fourth embodiment of the invention.

### DETAILED DESCRIPTION OF THE INVENTION

The invention discloses a non-invasive detecting apparatus and an operating method thereof. In practical applications, the non-invasive detecting apparatus of the invention can be applied in medical wearable non-invasive diagnosis. Because the non-invasive detecting apparatus of the invention can be worn on the hands of the operator, it can be easily operated and smoothly detect the detected region with high surface relief without changing detector.

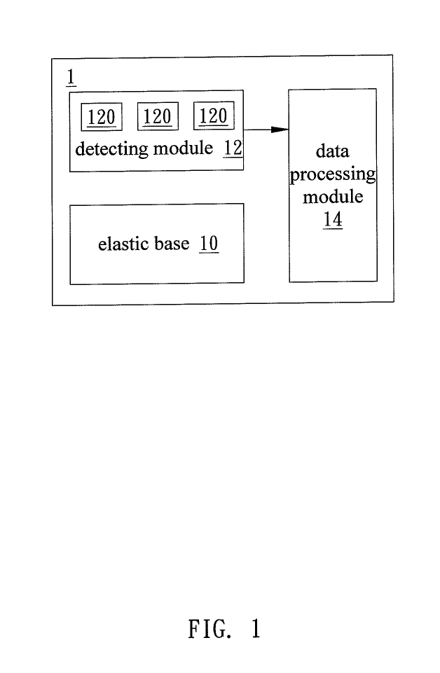

A first embodiment of the invention is a non-invasive detecting apparatus. Please refer to FIG. 1. FIG. 1 illustrates a function block diagram of the non-invasive detecting apparatus in this embodiment. As shown in FIG. 1, the non-invasive detecting apparatus 1 includes an elastic base 10, a detecting module 12, and a data processing module 14. Wherein, the detecting module 12 includes at least one detecting unit 120. In fact, the number of the detecting units 120 in the detecting module 12 can be determined based on practical needs without any specific limitations. The detecting module 12 is disposed on the elastic base 10. The data is transmitted between the detecting module 12 and the data processing module 14 through a wire way or a wireless way.

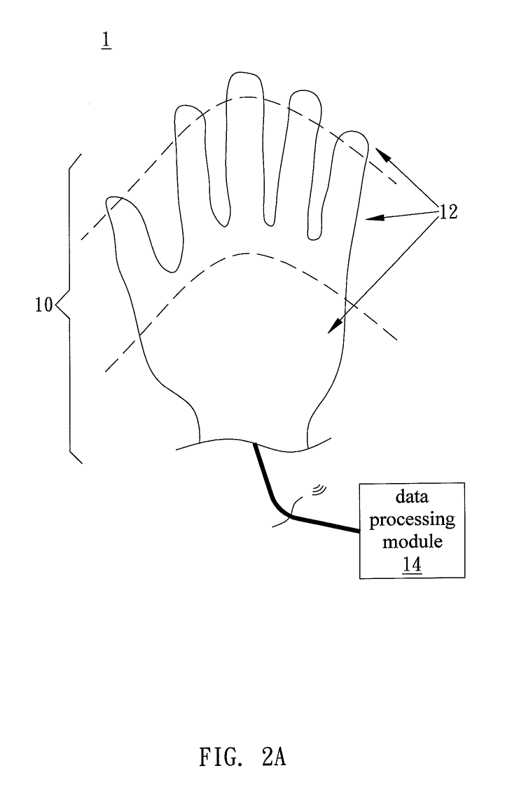

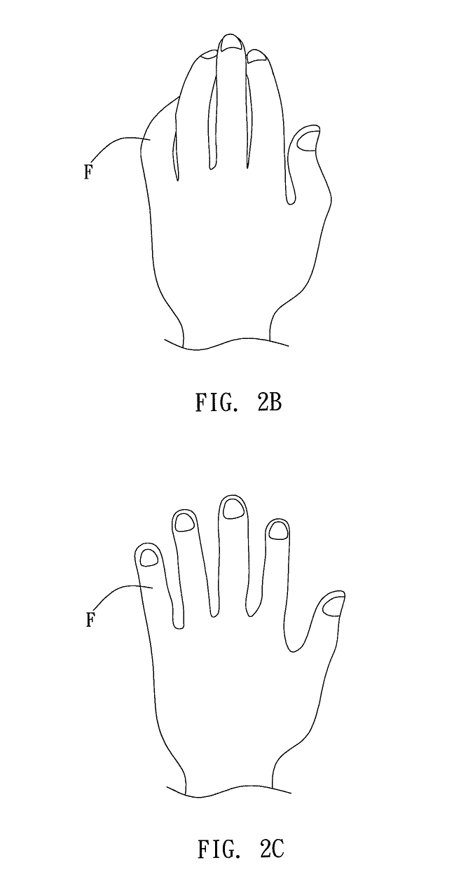

In practical applications, the non-invasive detecting apparatus 1 can be designed as a form which is wearable on the hands (e.g., the glove), but not limited to this. Please refer to FIG. 2A. FIG. 2A illustrates a schematic diagram of the non-invasive detecting apparatus 1 designed in a form of glove. As shown in FIG. 2A, the non-invasive detecting apparatus 1 in the form of glove can be directly worn on the right hand or the right hand. When the user moves the hand wearing the non-invasive detecting apparatus 1 to the surface of the detected region, the detecting units 120 disposed on the elastic base 10 at the finger end, the finger body, or the palm will perform deep detection on the tissue under the surface of the detected region to obtain a detection information related to the tissue under the surface of the detected region.

It should be noticed that the elastic base 10 of the non-invasive detecting apparatus 1 has the extending flexibility; therefore, the user can do detecting motions of bending finger (FIG. 2B), extending finger (FIG. 2C), or shifting finger (FIG. 2D) respectively to change the positions of the detecting units 120 on the elastic base 10 or the relative positions of the detecting units 120 relative to the elastic base 10 to change the size of the detected region of the detecting unit 120. Compared to the fixed design of detecting units in the detector of the prior art, the detecting units 120 of the detecting module 12 in the non-invasive detecting apparatus 1 of the invention has advantages of high efficiency and high using flexibility.

In this embodiment, for convenient, the non-invasive detecting apparatus 1 can store at least one default detecting motion and a position compensating information corresponding to the at least one default detecting motion in advance. In practical operation, after the non-invasive detecting apparatus 1 starts a default detection mode, the hand wearing the non-invasive detecting apparatus 1 only needs to do a default detecting motion to the surface of the detected region according to the operation guidebook, the data processing module 14 of the non-invasive detecting apparatus 1 can receive the detection results and the position compensating information corresponding to the default detecting motion transmitted from the detecting units 120 of the detecting module 12. Then, the data processing module 14 will adjust the detection result according to the position compensating information, so that the detection result will be not distorted due to the position changes of the detecting units 120 of the detecting module 12 or the relative position changes of the detecting units 120 relative to the elastic base 10. For example, the default detecting motion can be the finger F bending detecting motion mode shown in FIG. 2B, the finger F extending detecting motion mode shown in FIG. 2C, or other default detecting motion modes, if the non-invasive detecting apparatus 1 can judge the default detecting motion mode and transmit the corresponding position compensating information to the data processing module 14, there is no specific limitations.

In fact, after the at least one detecting unit 120 of the detecting module 12 obtains the detection information related to the tissue under the surface of the detected region, the at least one detecting unit 120 of the detecting module 12 can transmit the detection information to the data processing module 14 in a wire way or a wireless way.

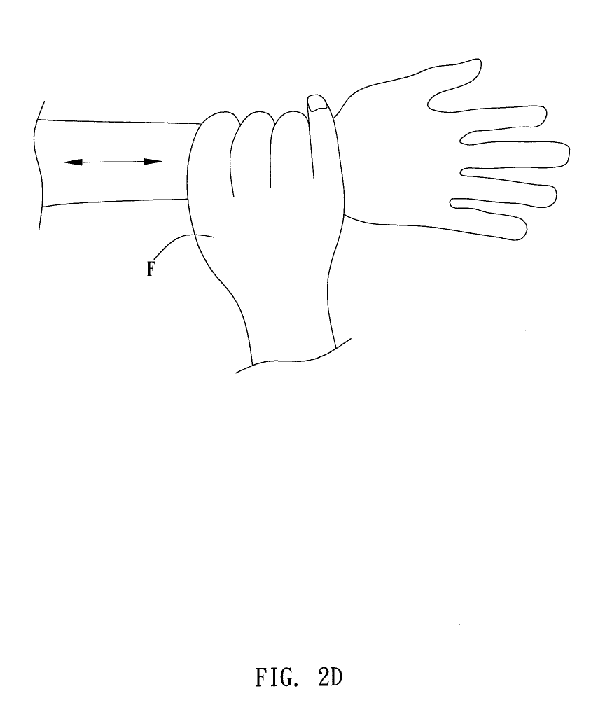

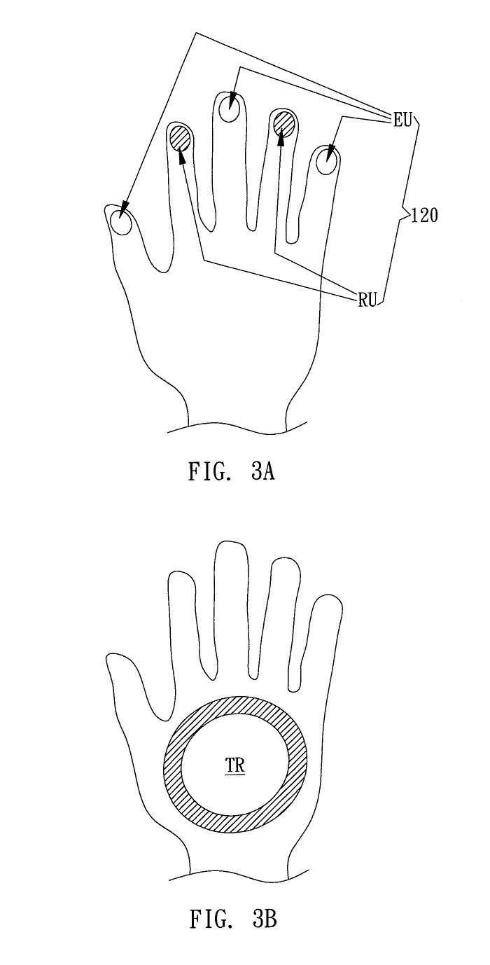

Please refer to FIG. 3A and FIG. 3B. FIG. 3A and FIG. 3B illustrate schematic diagrams of the different types of detecting units 120 of the detecting module 12 respectively. As shown in FIG. 3A and FIG. 3B, the detecting unit 120 of the detecting module 12 is formed by an emitting unit EU and a receiving unit RU, and the emitting unit EU and the receiving unit RU can be separated from each other (as shown in FIG. 3A) or integrated into a transceiver TR (as shown in FIG. 3B). In fact, the form, size, and position of the emitting unit EU and the receiving unit RU can be changed based on practical needs. For example, the form can be cycle, ellipse, strip, or other geometry without specific limitations.

In this embodiment, the at least one detecting unit 120 of the detecting module 12 uses a non-invasive detecting technology to perform the detection. In fact, the non-invasive detecting technology can be an ultrasound detecting technology, an optical detecting technology, an electrical detecting technology, or a magnetic detecting technology, it has no specific limitations. For example, except the ultrasound detecting technology, the at least one detecting unit 120 of the detecting module 12 can also use the optical coherence tomography (OCT) technology to perform deep detection on the tissue under the detected region. Its vertical detecting depth is about 2-3 mm, and the wavelength of the light it uses can be 1300 nm or 849 nm, but not limited to this.

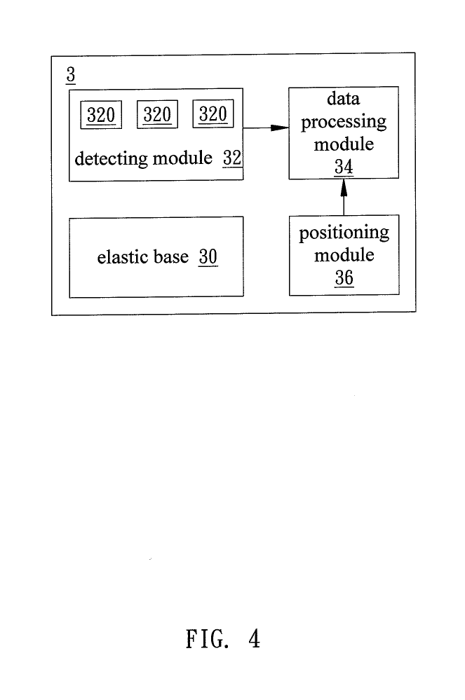

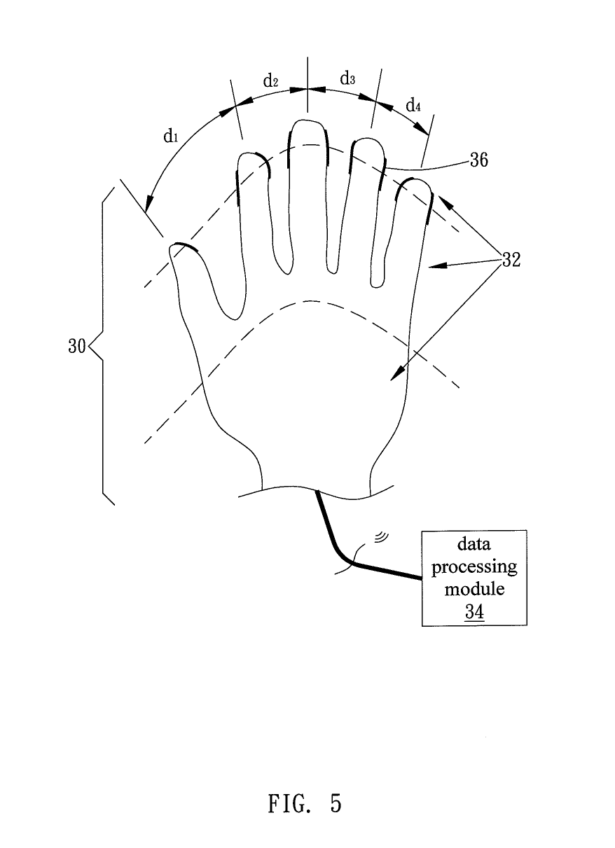

A second embodiment of the invention can be also a non-invasive detecting apparatus. Please refer to FIG. 4 and FIG. 5. FIG. 4 and FIG. 5 illustrate a function block diagram and the schematic diagram of the non-invasive detecting apparatus in this embodiment. As shown in FIG. 4 and FIG. 5, the non-invasive detecting apparatus 3 includes an elastic base 30, a detecting module 32, a data processing module 34, and a positioning module 36. Wherein, the detecting module 32 includes at least one detecting unit 320. The detecting module 32 is disposed on the elastic base 30. The positioning module 36 is coupled to the data processing module 34. The data is transmitted between the detecting module 32 and the data processing module 34 through a wire way or a wireless way.

It should be noticed that the difference between the non-invasive detecting apparatus 3 of this embodiment and the non-invasive detecting apparatus 1 of the above-mentioned first embodiment is that the non-invasive detecting apparatus 3 further includes the positioning module 36. The positioning module 36 is used for detecting a position of the at least one detecting unit 320, and generating a position compensating information to the data processing module 34 according to a position change of the at least one detecting unit 320 (e.g., the detecting unit is bent) or a relative position change of the at least one detecting unit 320 relative to the elastic base 30 (e.g., the detecting unit is shifted). For example, the positioning module 36 can position to obtain the distances d1˜d4 among the detecting units 320 to generate the position compensating information. Then, the data processing module 34 will adjust the detection result according to the position compensating information, so that the detection result obtained by the non-invasive detecting apparatus 3 will be not distorted due to the position changes of the detecting units 320 of the detecting module 32 or the relative position changes of the detecting units 320 relative to the elastic base 30.

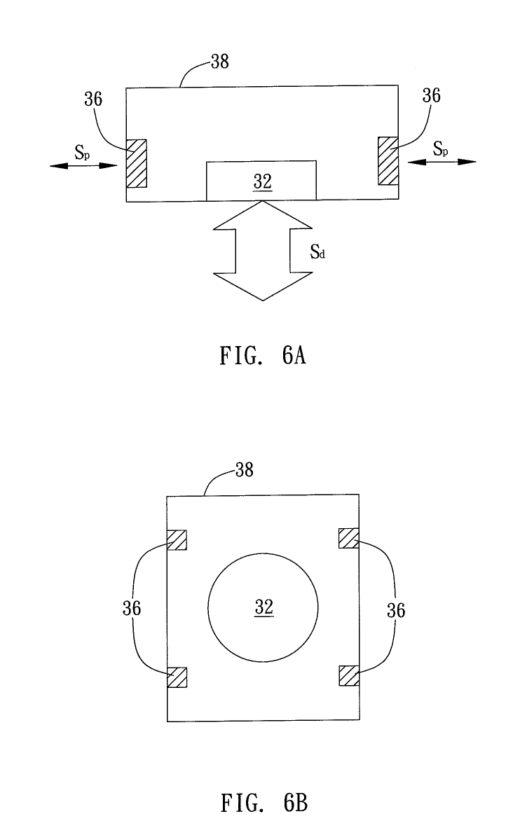

In practical applications, the positioning module 36 and the at least one detecting unit 320 of the detecting module 32 can be integrated. Please refer to FIG. 6A and FIG. 6B. FIG. 6A and FIG. 6B illustrate a side-view and a bottom-view of the positioning module 36 and the detecting unit 320 of the detecting module 32 integrated in the transceiver interface unit 38. As shown in FIG. 6A and FIG. 6B, the positioning module 36 is disposed on a side surface of the transceiver interface unit 38 and the detecting unit 320 of the detecting module 32 is disposed on a bottom surface of the transceiver interface unit 38. Therefore, the path of emitting and receiving the positioning signal Sₚ of the positioning module 36 is along the side surface direction of the transceiver interface unit 38, and the path of emitting and receiving the detecting signal S_{d} of the at least one detecting unit 320 of the detecting module 32 is along the bottom surface direction of the transceiver interface unit 38. It can avoid the interference between the signals emitted or received by the positioning module 36 and the detecting unit 320 of the detecting module 32, so that the accuracy of the final detection result will not be affected.

It should be noticed that the type of integrating the positioning module 36 and the detecting module 32 is only an embodiment, the positioning module 36 and the detecting module 32 can also have other different integration types, not limited to this case.

In practical applications, before the non-invasive detecting apparatus 3 starts to use the at least one detecting unit 320 of the detecting module 32 to detect, the non-invasive detecting apparatus 3 will start the positioning module 36 at first, so that when the at least one detecting unit 320 of the detecting module 32 detects, the positioning module 36 will also detect the position of the at least one detecting unit 320. Once the positioning module 36 detects the position change of the at least one detecting unit 320 (e.g., the detecting unit 320 is bent) or a relative position change of the at least one detecting unit 320 relative to the elastic base 30 (e.g., the detecting unit 320 is shifted), the positioning module 36 will generate corresponding position compensating information to the data processing module 34. Then, the data processing module 34 will adjust the detection result according to the position compensating information. After the at least one detecting unit 320 of the detecting module 32 finishes the detection, the non-invasive detecting apparatus 3 will shut down the positioning module 36. In practical applications, the non-invasive detecting apparatus 3 can start the positioning module 36 and the default detection mode at the same time, or only start any one of the positioning module 36 and the default detection mode without any specific limitations.

A third embodiment of the invention is a non-invasive detecting apparatus operating method. In this embodiment, the non-invasive detecting apparatus includes an elastic base, a detecting module, and a data processing module. The detecting module includes at least one detecting unit. The number of the detecting unit can be determined based on practical needs without any specific limitations. The detecting module is disposed on the elastic base. The data is transmitted between the detecting module and the data processing module through a wire way or a wireless way. In this embodiment, for convenient, the elastic base can be worn on the hand of the operator, so that the position of the at least one detecting unit can be changed with the variation of a gesture or the moving of a palm or a finger. At this time, the size of the detected region that the at least one detecting unit detects the detected object will also changed with the variation of the position of the at least one detecting unit. In addition, the non-invasive detecting apparatus can store at least one default detecting motion and a position compensating information corresponding to the at least one default detecting motion in advance.

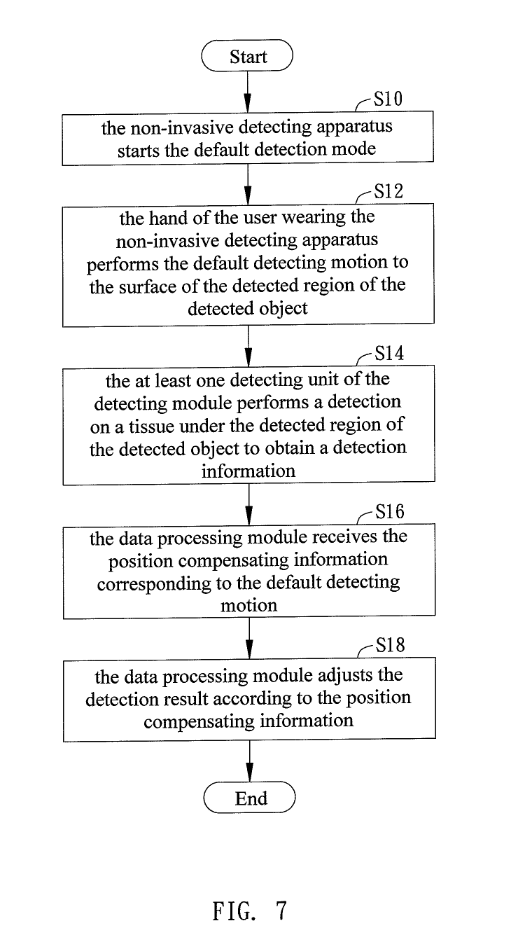

Please refer to FIG. 7. FIG. 7 illustrates the flowchart of the non-invasive detecting apparatus operating method in this embodiment. As shown in FIG. 7, at first, in step S10, the non-invasive detecting apparatus starts the default detection mode. Next, in step S12, the hand of the user wearing the non-invasive detecting apparatus performs the default detecting motion to the surface of the detected region of the detected object. Then, in step S14, the at least one detecting unit of the detecting module performs a detection on a tissue under the detected region of the detected object to obtain a detection information. At the same time, in step S16, the data processing module receives the position compensating information corresponding to the default detecting motion. Afterward, in step S18, the data processing module adjusts the detection result according to the position compensating information, so that the detection result will be not distorted due to the position changes of the detecting units of the detecting module or the relative position changes of the detecting units relative to the elastic base.

In this embodiment, the detecting units of the detecting module use a non-invasive detecting technology to perform the detection. In fact, the non-invasive detecting technology can be an ultrasound detecting technology, an optical detecting technology, an electrical detecting technology, or a magnetic detecting technology, it has no specific limitations. For example, except the ultrasound detecting technology, the detecting unit can also use the optical coherence tomography (OCT) technology to perform deep detection on the tissue under the detected region. Its vertical detecting depth is about 2-3 mm, and the wavelength of the light it uses can be 1300 nm or 849 nm, but not limited to this.

A fourth embodiment of the invention is a non-invasive detecting apparatus operating method. In this embodiment, the non-invasive detecting apparatus includes an elastic base, a detecting module, a data processing module, and a positioning module. The detecting module includes at least one detecting unit. The detecting module is disposed on the elastic base. The positioning module is coupled to the data processing module. The data is transmitted between the detecting module and the data processing module through a wire way or a wireless way.

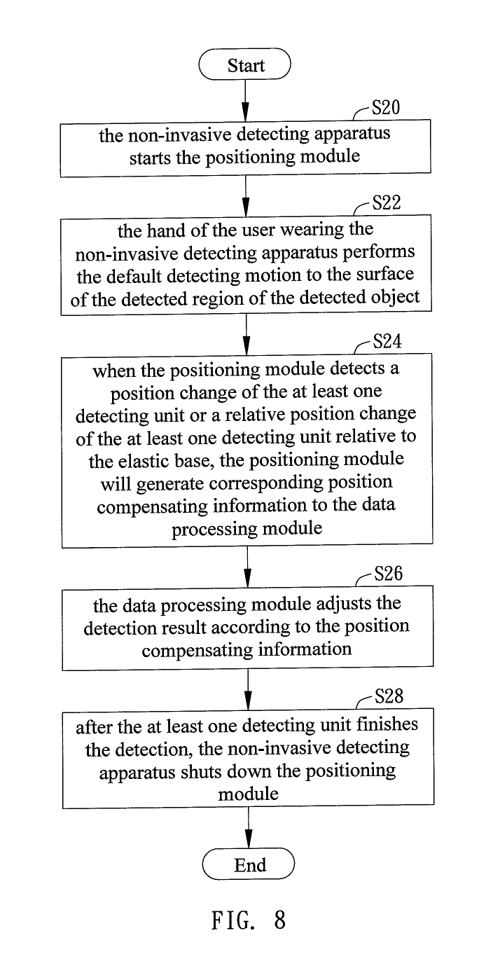

Please refer to FIG. 8. FIG. 8 illustrates the flowchart of the non-invasive detecting apparatus operating method in this embodiment. As shown in FIG. 8, at first, in step S20, the non-invasive detecting apparatus starts the positioning module. Next, in step S22, the hand of the user wearing the non-invasive detecting apparatus performs the default detecting motion to the surface of the detected region of the detected object. Then, in step S24, when the positioning module detects a position change of the at least one detecting unit or a relative position change of the at least one detecting unit relative to the elastic base, the positioning module will generate corresponding position compensating information to the data processing module. Afterward, in step S26, the data processing module adjusts the detection result according to the position compensating information, so that the detection result will be not distorted due to the position changes of the detecting units of the detecting module or the relative position changes of the detecting units relative to the elastic base. At last, in step S28, after the at least one detecting unit finishes the detection, the non-invasive detecting apparatus shuts down the positioning module.

Compared to the prior art, the non-invasive detecting apparatus and the operating method thereof disclosed in this invention can avoid the disadvantages of the conventional non-invasive detecting apparatus that various ultrasound detectors of different sizes should be prepared and only a partial region of the ultrasound detector can detect. Even the surface relief of the detected region is too large, since the non-invasive detecting apparatus of the invention can be worn on the hands of the operator, it can be easily operated and smoothly detect without changing detector. It can be also applied in large-area and multi-angles synchronous detection and different non-invasive detecting technologies.

In addition, because the detecting module in the non-invasive detecting apparatus of the invention is integrated with the elastic base, the operator can change the positions of the detecting units (e.g., bending) or the relative positions of the detecting units relative to the elastic base (e.g., shifting) to change the range covered by its detected area. Therefore, compared to the fixed design of detecting units in the detector of the prior art, the detecting module in the non-invasive detecting apparatus of the invention has advantages of high efficiency and high using flexibility.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A non-invasive detecting apparatus, comprising:
an elastic base;
a detecting module, disposed on the elastic base, the detecting module comprising at least one detecting unit used for performing a detection on a tissue under a detected region of a detected object to obtain a detection information; and
a data processing module, for analyzing and processing the detection information to generate a detection result.

2. The non-invasive detecting apparatus of claim 1, wherein the elastic base is worn on a hand of a user, and a position of the at least one detecting unit can be changed with the variation of a gesture or the moving of a palm or a finger.

3. The non-invasive detecting apparatus of claim 1, wherein the at least one detecting unit is formed by an emitting unit and a receiving unit, and the emitting unit and the receiving unit are integrated into a transceiver or separated from each other.

4. The non-invasive detecting apparatus of claim 1, further comprising:
a positioning module, coupled to the data processing module, the positioning module detecting a position of the at least one detecting unit, and generating a position compensating information to the data processing module according to a position change of the at least one detecting unit or a relative position change of the at least one detecting unit relative to the elastic base, the data processing module adjusting the detection result according to the position compensating information.

5. The non-invasive detecting apparatus of claim 4, wherein the positioning module and the at least one detecting unit of the detecting module are integrated.

6. The non-invasive detecting apparatus of claim 1, wherein the detecting module uses a non-invasive detecting technology to perform the detection, the non-invasive detecting technology is an ultrasound detecting technology, an optical detecting technology, an electrical detecting technology, or a magnetic detecting technology.

7. A method of operating a non-invasive detecting apparatus, the non-invasive detecting apparatus comprising an elastic base, a detecting module, and a data processing module, the detecting module comprising at least one detecting unit and being disposed on the elastic base, the method comprising steps of:
the at least one detecting unit of the detecting module performing a detection on a tissue under a detected region of a detected object to obtain a detection information; and
the data processing module analyzing and processing the detection information to generate a detection result.

8. The method of claim 7, wherein the elastic base is worn on a hand of a user, and a position of the at least one detecting unit can be changed with the variation of a gesture or the moving of a palm or a finger.

9. The method of claim 7, further comprising steps of:
detecting a position of the at least one detecting unit;
generating a position compensating information to the data processing module according to a position change of the at least one detecting unit or a relative position change of the at least one detecting unit relative to the elastic base; and
adjusting the detection result according to the position compensating information.

10. The method of claim 7, wherein the detecting module uses a non-invasive detecting technology to perform the detection, the non-invasive detecting technology is an ultrasound detecting technology, an optical detecting technology, an electrical detecting technology, or a magnetic detecting technology.
